(19) United States
(12) Patent Application Publication
(10) Pub. No.: US 2026/0321406 A1
(43) Pub. Date: Oct. 15, 2026

(54) DYNAMIC PERSONA SWITCHING IN A CONVERSATIONAL ARTIFICIAL INTELLIGENCE (AI) BASED AGENT BASED ON CONVERSATION ATTRIBUTES

(71) Applicant: HealthGPT, Inc. DBA Hippocratic AI, Palo Alto, CA (US)

(72) Inventors: Kriti Aggarwal, Redmond, WA (US); Subhabrata MUKHERJEE, Palo Alto, CA (US); Shanil Puri, Palo Alto, CA (US); Tanmay Laud, Palo Alto, CA (US); Woojeong Jin, Palo Alto, CA (US); Jan Schellenberger, Palo Alto, CA (US)

(73) Assignee: HealthGPT, Inc. DBA Hippocratic AI, Palo Alto, CA (US)

(21) Appl. No.: 19/718,319

(22) Filed: Jun. 24, 2026

Related U.S. Application Data
(63) Continuation-in-part of application No. 19/400,096, filed on Nov. 25, 2025.
Application No. 19/400,096 is a continuation-in-part of application No. 18/900,289, filed on Sep. 27, 2024.
Application No. 18/900,289 is a continuation of application No. 18/592,441, filed on Feb. 29, 2024, now Pat. No. 12,142,371.
(60) Provisional application No. 63/990,092, filed on Feb. 24, 2026.
(60) Provisional application No. 63/611,762, filed on Dec. 18, 2023.
(60) Provisional application No. 63/466,712, filed on May 15, 2023.

(51) Int. Cl. G16H 40/20; G10L 15/183; G10L 15/22; G10L 25/66; G10L 15/16; G10L 15/18; G10L 15/19

(52) U.S. Cl. CPC G16H 40/20; G10L 15/183; G10L 15/22; G10L 25/66; G10L 15/16; G10L 15/1822; G10L 15/19

(57) ABSTRACT

In some cases, an artificial intelligence agent engages in a conversation with a human and receives a first audio input from the human. The artificial intelligence agent determines a first set of attributes of the first audio input and encodes the first set of attributes to create a first set of tags, where individual attributes of the first set of attributes have a corresponding tag in the first set of tags. Based on the first set of tags, a persona of the artificial intelligence agent is modified to create a first modified agent having a first modified persona. The first modified agent having the first modified persona provides a first response to the human.

### PRIORITY DATA

The present non-provisional patent application claims priority to and the benefit from (1) U.S. Application 63/990,092 filed on Feb. 24, 2026, and (2) U.S. Application 19/400,096 filed on Nov. 25, 2025, which are incorporated herein by reference in their entirety and for all purposes as if completely and fully set forth herein.

### BACKGROUND OF THE TECHNOLOGY DISCLOSED

### Field of the Technology Disclosed

The technology disclosed relates to artificial intelligence type computers and digital data processing systems and corresponding data processing methods and products for emulation of intelligence (i.e., knowledge-based systems, reasoning systems, and knowledge acquisition systems); and including systems for reasoning with uncertainty (e.g., fuzzy logic systems), adaptive systems, machine learning systems, and artificial neural networks. In particular, the technology disclosed relates generally to systems and techniques that determine attributes associated with audio input provided by a human and use the attributes to modify an artificial intelligence (AI) based agent to dynamically create a modified persona.

### Description of the Related Art

Current artificial intelligence (AI) virtual assistants (including chat bots), that engage in a conversation with a human, have a variety of issues. For example, current conversational systems use a fixed persona that does not change during a conversation with a human or from one conversation to another. For example, if a human engages in a first conversation with an AI virtual assistant and later engages in a second conversation, the AI virtual assistant will engage in the second conversation in a manner similar to how the AI virtual assistant engaged with the human in the first conversation, even if the human is speaking with more urgency in the second conversation. The AI virtual assistants are incapable of adapting to shifts in the user’s emotional state, task needs, or conversational flow. This rigidity produces interactions that feel repetitive, mismatched, or inappropriate. This is especially notable in sensitive domains such as healthcare.

### SUMMARY OF THE TECHNOLOGY DISCLOSED

This Summary provides a simplified form of concepts that are further described below in the Detailed Description. This Summary is not intended to identify key or essential features and should therefore not be used for determining or limiting the scope of the claimed subject matter.

In some cases, an artificial intelligence agent engages in a conversation with a human and receives a first audio input from the human. The artificial intelligence agent determines a first set of attributes of the first audio input and encodes the first set of attributes to create a first set of tags, where individual attributes of the first set of attributes have a corresponding tag in the first set of tags. Based on the first set of tags, a persona of the artificial intelligence agent is modified to create a first modified agent having a first modified persona. The first modified agent having the first modified persona provides a first response to the human.

### BRIEF DESCRIPTION OF THE DRAWINGS

A more complete understanding of the present disclosure may be obtained by reference to the following Detailed Description when taken in conjunction with the accompanying Drawings. In the figures, the left-most digit(s) of a reference number identifies the figure in which the reference number first appears. The same reference numbers in different figures indicate similar or identical items.

FIG. 1 is a block diagram of a system illustrating large language models (LLMs) to perform specialized healthcare-related functions, according to some implementations.

FIG. 2 is a block diagram illustrating an artificial intelligence (AI) architecture, according to some implementations.

FIG. 3 is a block diagram of a system that includes a low-latency conversational artificial intelligence (AI) architecture with a parallelized in-depth analysis and feedback loop, according to some implementations.

FIG. 4 is a block diagram of a system to determine attributes of human input and modify a persona of an AI-based agent based on the attributes, according to some implementations.

FIG. 5 is a flowchart of a process that includes determining attributes of an audio input received from a human, according to some implementations.

FIG. 6 is a flowchart of a process to train a machine learning algorithm, according to some implementations.

FIG. 7 illustrates an example configuration of a computing device that can be used to implement the systems and techniques described herein.

### DETAILED DESCRIPTION

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections – these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

The systems and techniques described herein provide for real-time, adaptive, dynamic persona control of conversational AI agents, incorporating user archetypes, voice-derived conversational cues, and contextual urgency signals. The system and techniques determine attributes of audio input provided by a human during a conversation with an AI agent, create tags based on the attributes, and use the tags to dynamically and adaptively adjust the AI agent’s personality, tone, interaction style, and conversational strategy during the conversation. By integrating acoustic features (e.g., tempo, pauses, intonation, pitch, and the like), sentiment, urgency level, and archetype-based preferences, the system is able to modify the persona of the agent seamlessly to enable responses that feel relevant, attuned, and adaptive. Real world testing has demonstrated an up to 20% improvement in call completion rates, highlighting the measurable impact of the systems and techniques described herein.

The persona change may mimic one or more of the attributes of the human’s input. For example, assume the human speaks in brief, to-the-point sentences or the human says “I am in a hurry right now and just wanted to confirm <X>.” In response, the AI agent may be modified to a concise (non-verbose) persona. The concise persona may include speaking slightly faster (shorter gaps between words), using shorter words, and so on to keep the conversation relatively brief. In this example, the concise persona matches the brevity of the human’s speech.

The persona change may be designed to address one or more of the attributes of the human’s input. For example, the human input may have attributes (e.g., higher pitched voice, sounds of agitation or crying, or the like) that indicate that the human is feeling anxious or upset. In response, the AI agent may be modified to use a calming persona that speaks (i) at a slower (than normal) pace, (ii) at a lower (than normal) pitch, (iii) using a melodic intonation, or any combination thereof.

Thus, a system enables AI-agents to dynamically (during a conversation) adapt to attributes of a human’s speech, including communication style, emotional state, and situational urgency. The systems and techniques enable AI-agents to determine an archetype of the human, adjust tone and/or behavior based on real-time voice cues, detect urgency and modify the AI agent’s persona accordingly, dynamically blend multiple personas, and maintain coherency during persona shifts across multi-turn conversations. In contrast, conventional AI-agents use static, predefined personas, fixed prompts, and the like that result in rigid, mismatched, or ineffective interactions that may be counterproductive in task-oriented voice calls such as healthcare triage, benefits navigation, or appointment scheduling. The systems and techniques described herein overcome these shortcomings through a tag-based persona system that adapts continuously to the human’s behavior, voice signals, and conversation trajectory.

A conversational AI system is trained to identify attributes of a human’s speech, create tags associated with each of the attributes, and dynamically, during a conversation between the human and the AI agent, modify an AI agent’s persona based on the tags. The tags may represent human speech attributes such as, for example, tone (e.g., formal, casual, warm, authoritative, and the like), personality traits (e.g., witty, concise, empathetic, familiar, tangent-oriented, and the like), conversation style (e.g., slow-paced, fast-paced, highly structured, and the like), behavioral cues (e.g., reassuring, directive, reflective, and the like), and an urgency level.

The systems and techniques may use archetype profiles (of humans) derived from demographics, call context, prior interactions, behavioral signals, and the like. Voice cues, including hesitation, rising pitch, breathiness, affective energy, speech rate, and interruptions may be used to generate tags. For example, urgency signals may be detected based on linguistic markers (e.g., choice of words that are spoken) and acoustic markers (e.g., speed of speaking, length of pauses, pitch, and the like). The systems and techniques may determine conversation state indicators, such as, for example, frustration patterns, confusion, disengagement, and pacing. Together, these features guide real-time persona modifications that improve the user experience and help the AI agent with task completion.

A tag repository may be used to store human speech attributes and their corresponding tags. The tag repository may be updated and expanded over time to add new attributes and their corresponding tags. The tag repository may be a structured library of persona attributes, tonal styles, and behavioral modifiers, including, for example, an empathy level, conciseness or elaboration, familiarity and warmth, more structured or less structured conversational pacing, urgency-handling modes, domain expertise modulation, wit, tangent behavior, and knowledge density. Each tag in the tag repository is designed to modify a particular aspect of the AI agent’s persona.

A persona modifier module may map incoming (e.g., most recently determined) tags to latent behavioral representations and apply them in real time to modify the persona of an AI agent. The persona modifier module may perform tag stacking (modifying a persona based on two or more tags), tag blending (modifying a persona based on blending two or more tags), tag priority (multiple tags may be weighted based on priority), decay of old tags (older tags are aged out), and real time persona switching without disrupting coherence when switching from one persona to another persona.

A dynamic persona handler may determine when and how a persona may shift (from one persona to another persona) based on:
(1) voice cues, such as a tone of voice, a tempo of speech, interjections, and stress markers, such as sighs,
(2) urgency cues, such as rapid speech, repeated queries, and explicitly expressed time constraints,
(3) engagement cues, such as silence patterns and disengagement markers,
(4) user archetype detection, such as cautious, assertive, novice, expert, anxious, and the like, and (5) conversation goals, such as escalation, de-escalation, clarification, and task completion.

When a conversation between a human and an AI agent is initiated, the system may
(1) determine an initial user archetype (e.g., rushed caller, confused caller, elderly caller, highly knowledgeable caller, or the like),
(2) determine an initial set of voice cues, and
(3) select an appropriate base persona. During the conversation between the human and the AI agent, the tags may be updated multiple times during the conversation. For example, the tags may be updated when the human has stopped speaking (completed a turn), after each sentence, after each word, or any combination thereof. As the conversation progresses, the system may
   (i) extract a set of current voice cues (pitch, stress, speed, pauses, and the like),
   (ii) determine an urgency level and identify frustration patterns (based on one or more words or phrases, pitch, non-verbal cues, and the like),
   (iii) determine the human’s engagement, and adjust tags and modify the persona to maintain alignment with the human.

The systems and techniques maintain consistency within a conversation to avoid an abrupt persona shift, unless triggered by one or more high-urgency signals. The persona may be reset between conversations (e.g., a first conversation occurs on a first date and a second conversation occurs on a second date) to avoid long-term bias.

### Examples

When the system determines that a human sounds confused or overwhelmed, it causes the system to modify the persona to increase empathy, provide a response at a slow pace, or use reassuring words and phrases. When the system determines a sense of urgency, e.g., faster speech, a higher pitched voice, or the like, the system may modify the persona to provide a more concise and direct speech persona. When the system identifies customer frustration cues, such as a sharp tone, faster speech, interruptions, or the like, the system may modify the persona to a calming persona, more structured instructions, less elaboration, and higher clarity. When the system determines that a human is knowledgeable, the persona may be modified to a more concise, expert-level persona. Test results using a dynamically adaptive persona for AI agents as described herein has shown an approximately 20% improvement in call completion rates.

Thus, the systems and techniques describe real-time persona modification in conversational agents, including:
(i) determining speech attributes (e.g., voice cues, urgency signals, and the like) associated with speech input from a human that is in a conversation with an AI agent,
(ii) determining tags based on the attributes,
(iii) modifying a persona of the AI agent based on the tags, and (iv) dynamically adjusting the persona based on the conversation context, user archetype, and detected voice-derived cues. The tags include behavioral traits selected from wit, conciseness, familiarity, empathy, urgency mode, pacing style, and knowledge density. The persona adjustments may be made in response to contextual cues, including, for example, user sentiment, detected stress markers, engagement patterns, silence timing, speech rate, and acoustic affect signals. The AI agent’s persona is modified by the tags. The system is able to improve task completion metrics by dynamically aligning persona with user archetype, voice cues, and urgency signals, resulting in measurable improvements of up to 20% increase in call completion.

As a first example, an artificial intelligence agent executed by one or more processors engages in a conversation with a human. The artificial intelligence agent receives a first audio input from the human. The audio input may be provided to an automatic speech recognition (ASR) module and a text output that is derived from the audio input may be received from the automatic speech recognition module. The one or more processors determine a first set of attributes of the first audio input and encode the first set of attributes to create a first set of tags in which individual attributes of the first set of attributes have a corresponding tag in the first set of tags. For example, the one or more attributes of the audio input may include: a voice cue, a personality trait, an urgency cue, a conversation style, a conversation state, a tone, or any combination thereof. The one or more processors, based on the first set of tags, modify a persona of the artificial intelligence agent to create a first modified agent having a first modified persona. The first modified agent having the first modified persona, provides a first response to the human. The one or more processors may detect an urgency signal in the first audio input based at least in part on: (i) a linguistic marker comprising a particular word in the first audio input, (ii) an acoustic marker comprising at least one of:
(a) a speed of speech included in the first audio input,
(b) a length of one or more pauses in the first audio input,
(c) a pitch of one or more words in the first audio input, or
(d) any combination thereof, or (iii) both the linguistic marker and the acoustic marker. The first modified agent having the first modified persona may receive a second audio input from the human, may determine a second set of attributes of the second audio input, and may encode the second set of attributes into a second set of tags. In some cases, the first modified persona of the first modified agent may be modified based on the second set of tags to create a second modified agent having a second persona and the second modified agent, having the second persona, may provide a second response to the human. During the conversation with the human, the one or more processors may maintain coherency when transitioning from the first modified agent having the first modified persona to the second modified agent having the second modified persona. The first response provided by the first modified agent having the first modified persona may use a first cadence and the second response provided by the second modified agent having the second persona may use a second cadence in which at least one word has:
   (i) a different pitch than the first cadence,
   (ii) a different volume than the first cadence,
   (iii) a different length of a pause after the at least one word than the first cadence, or (iv) any combination thereof.

As a second example, a server includes one or more processors and one or more computer-readable storage media to store instructions executable by the one or more processors to perform various operations. The operations include engaging, by an artificial intelligence agent, in a conversation with a human. The operations include receiving, by the artificial intelligence agent, a first audio input from the human. The operations include determining a first set of attributes of the first audio input. For example, the first set of attributes may include: a tone (such as formal, casual, warm, authoritative, or any combination thereof), a personality trait (such as witty, concise, empathetic, familiar, tangent-oriented, or any combination thereof), a conversation style (such as slow-paced, fast-paced, highly structured, or any combination thereof), a behavioral cue (such as reassuring, directive, reflective, or any combination thereof), an urgency level (such as urgent or non-urgent), or any combination thereof. The operations include encoding the first set of attributes to create a first set of tags, where individual attributes of the first set of attributes having a corresponding tag in the first set of tags. The operations include modifying, based on the first set of tags, a persona of the artificial intelligence agent to create a first modified agent having a first modified persona. The operations include providing, by the first modified agent having the first modified persona, a first response to the human. The modified persona may include: a verbose persona that provides additional details about one or more topics in the conversation, a concise persona that does not provide the additional details about the one or more topics in the conversation, an empathetic persona that mirrors a speaking style of the human, a calming persona that is designed to invoke a calming response in the human, or any combination thereof. The operations may include determining a conversation state that includes one of: engagement, disengagement, frustration, or confusion. The operations may include receiving, by the first modified agent having the first modified persona, a second audio input from the human, determining a second set of attributes of the second audio input, encoding the second set of attributes into a second set of tags, modifying, based on the second set of tags, the first modified persona of the first modified agent to create a second modified agent having a second persona, and providing, by the second modified agent having the second persona, a second response to the human. During the conversation with the human, the operations may include maintaining coherency when transitioning from the first modified agent having the first modified persona to the second modified agent having the second modified persona. The first response provided by the first modified agent having the first modified persona may use a first cadence and the second response provided by the second modified agent having the second persona may use a second cadence in which at least one word has: a different pitch than the first cadence, a different volume than the first cadence, a different length of a pause after the at least one word than the first cadence, or any combination thereof.

As a third example, a non-transitory memory device is used to store instructions executable by one or more processors to perform various operations. The operations include engaging, by an artificial intelligence agent, in a conversation with a human. The operations include receiving, by the artificial intelligence agent, a first audio input from the human. The operations include determining a first set of attributes of the first audio input. The operations include encoding the first set of attributes to create a first set of tags, where individual attributes of the first set of attributes having a corresponding tag in the first set of tags. The operations include modifying, based on the first set of tags, a persona of the artificial intelligence agent to create a first modified agent having a first modified persona. The operations include providing, by the first modified agent having the first modified persona, a first response to the human. The modified persona may include at least one of: an empathy level that is different from the persona of the artificial intelligence agent, a conciseness level that is different from the persona of the artificial intelligence agent, a familiarity level that is different from the persona of the artificial intelligence agent, a structured speech level that is different from the persona of the artificial intelligence agent, an urgency-handling mode that is different from the persona of the artificial intelligence agent, a knowledge density level that is different from the persona of the artificial intelligence agent, or any combination thereof. The operations may include determining one or more voice cues in the audio input including one or more of: an amount of hesitation between words spoken in the audio input, a rising pitch between a word and a subsequent word in the audio input, an amount of breath noise detected in the audio input, a speech rate of the words spoken in the audio input, or any combination thereof. The operations may include receiving, by the first modified agent having the first modified persona, a second audio input from the human, determining a second set of attributes of the second audio input, encoding the second set of attributes into a second set of tags, modifying, based on the second set of tags, the first modified persona of the first modified agent to create a second modified agent having a second persona, and providing, by the second modified agent having the second persona, a second response to the human. The second response provided by the second modified agent having the second persona includes at least one word having: a different pitch than words included in the first response; a different volume than the words included in the first response; a different length of a pause after the at least one word than pauses included in the first response; or any combination thereof. The operations may include detecting an urgency signal in the first audio input based at least in part on: (1) a linguistic marker comprising a particular word in the first audio input, (2) an acoustic marker comprising at least one of: (i) a speed of speech included in the first audio input, (ii) a length of one or more pauses in the first audio input, (iii) a pitch of one or more words in the first audio input, or (iv) any combination thereof, or (3) both the linguistic marker and the acoustic marker.

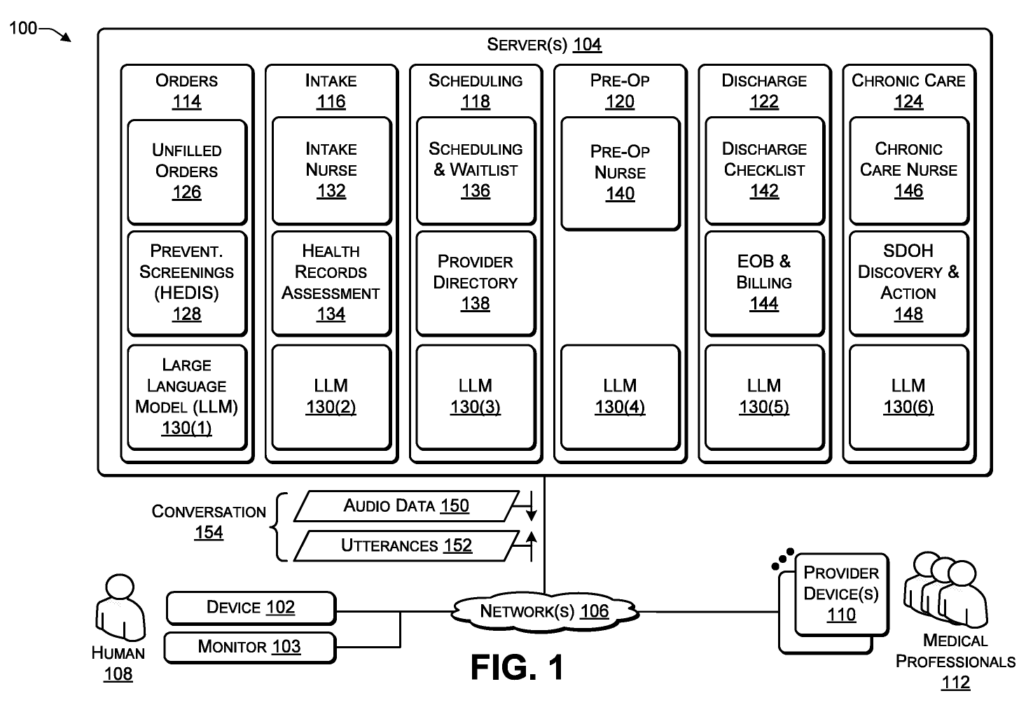

FIG. 1 is a block diagram of a system 100 illustrating large language models (LLMs) to perform specialized healthcare-related functions (e.g., roles), according to some implementations. The system 100 includes a device 102 connected to one or more servers 104 via one or more networks 106. The device 102 may be, for example, a smart phone, or another type of user device associated with a human 108. For example, the human 108 may be a current patient, a past patient, or a potential (future) patient. In some cases, the device 102 may be linked (“paired”) with a monitoring device (“monitor”) 103, such as a smart watch, a continuous glucose monitor (CGM), or another type of device that is capable of providing biometric readings associated with the human 108 to the device 102.

One or more provider devices 110 may be connected to the network 106. The provider devices 110 may be used by one or more medical professionals 112 (e.g., medical technicians, nurses, nurse practitioners, doctors, and the like) associated with a medical provider.

The server 104 may host multiple AI-based virtual assistants, such as the large language model (LLM) 130 virtual assistants. The virtual assistants LLM 130(1) to 130(6) shown in FIG. 1 each perform a particular healthcare-related role. Six LLM 130 are shown purely for illustration purposes. It should be understood that the number of virtual assistants LLM 130 may vary depending on how the healthcare functions (roles) are divided. The division of the healthcare functions influences the algorithm design, training data, and other details associated with the virtual assistants 130. A different division of the healthcare functions may result in fewer or more than six virtual assistants LLM 130. For illustration purposes, the six functions (roles) include orders 114, intake 116, scheduling 118, pre-op 120, discharge 122, and chronic care 124.

The orders 114 role may include the LLM 130(1) engaging in a conversation 154 with the human 108 to resolve unfilled orders 126. Unfilled orders 126 refers to prescribed medications that the human 108 has not picked up, prescribed medical procedures or follow-up appointments that the human 108 has not scheduled, and so on. The orders 114 role may include the LLM 130(1) entering into a conversation 154 with the human 108 to perform preventative screenings 128, including gathering data relative to Healthcare Effectiveness Data and Information Set (HEDIS) calculations.

The conversation 154 may include audio data 150 output by one of the LLM 130 and one or more utterances 152 from the human 108. The human 108 may speak the utterances 152 into a microphone of the device 102 and the device 102 may send the utterances 152 to the LLM 130 over the network 106. The LLM 130 may provide audio data 150 over the network 106 for playback by the device 102 to the human 108. The conversation 154 may be initiated by the LLM 130 or by the human 108.

The intake 116 role may include the LLM 130(2) engaging in the conversation 154 to perform the role of an intake nurse 132. For example, the conversation 154 may include the LLM 130(2) providing the human 108 with appointment-related information, such as a location and date and time associated with the appointment. The intake 116 role may include the LLM 130(2) engaging in the conversation 154 to perform a Health Records Assessment (HRA), determine a Risk Adjustment Factor (RAF), or the like.

The scheduling 118 role may include the LLM 130(3) engaging in the conversation 154 with the human 108 to perform scheduling and waitlisting related actions. For example, the LLM 130(3) may schedule the human 108 for an appointment and/or waitlist the human 108 for a consultation, surgery, or another type of appointment. The scheduling 118 role may include the LLM 130(3) engaging in the conversation 154 with the human 108 to access a provider directory to select a provider for the human 108. For example, if the human 108 has been asked (e.g., by a primary care physician) to have a consultation with a specialist, then the LLM 130(3) may identify a particular specialist in the provider directory 138 and schedule the human 108’s consultation with the particular specialist.

The pre-op 120 role may include the LLM 130(4) engaging in the conversation 154 and taking on the role of a pre-op nurse 140 to go through a pre-op checklist with the human 108. For example, the pre-op checklist may include various pre-op do’s and don’ts, such as what to eat, what not to eat (e.g., avoid solid foods at least 24 hours prior to a colonoscopy, avoid eating 12 hours prior to a blood glucose test, etc.), what medications to take, what medications to avoid taking (e.g., no beta blockers prior to a treadmill stress test), type of clothing to wear (e.g., loose clothing prior to a treadmill stress test), and so on.

The discharge 122 role may include the LLM 130(5) engaging in the conversation 154 to go over a discharge checklist 142. For example, the discharge checklist 142 may include describing what to do and what not do (e.g., don’t perform certain action for a particular period of time after certain types of surgery), reviewing medications to take (e.g., take X every day, take Y as needed, and the like), highlighting warning signs (e.g., call medical provider immediately if the human 108 experiences particular symptoms, such as shortness of breath, dizziness, blurry vision, or the like), making follow-up appointments, and the like. The discharge 122 role may include the LLM 130(5) providing information regarding insurance coverage, explanation of benefits (EOB), and other billing-related information (e.g., amount that insurance should cover, amount that the human is responsible for paying, and so on).

The chronic care 124 role may include the LLM 130(6) performing the role of a chronic care nurse when engaging in the conversation 154 with the human 108. For example, the chronic care 124 role may include reviewing medical records and test results, and providing information regarding self-management, such as diet/nutrition suggestions, exercise suggestions, advice on managing prescription regimens, and information regarding monitoring symptoms. The chronic care 124 role may include the LLM 130(6) engaging in the conversation 154 to determine social determinants of health (SDOH), which are the non-medical factors that influence health outcomes. SDOH include the conditions in which people are born, grow, work, live, and age, and the systems shaping the conditions of daily life, such as economic policies, development agendas, social norms, social policies, and political systems.

Thus, a medical provider may deploy AI engines, in the form of a large language model (LLM) or similar AI, to perform specialized health-related tasks. Each AI engine may be designed and trained for a particular purpose, such as screening, intake, scheduling, pre-op, discharge, chronic care, and the like. The advantages of using AI engines include (i) reducing costs compared to using a human, (ii) freeing up humans to perform in-person functions (roles), (iii) performing the health-related tasks at times that are convenient, including outside regular business hours, and (iv) reducing human error when performing the health-related tasks.

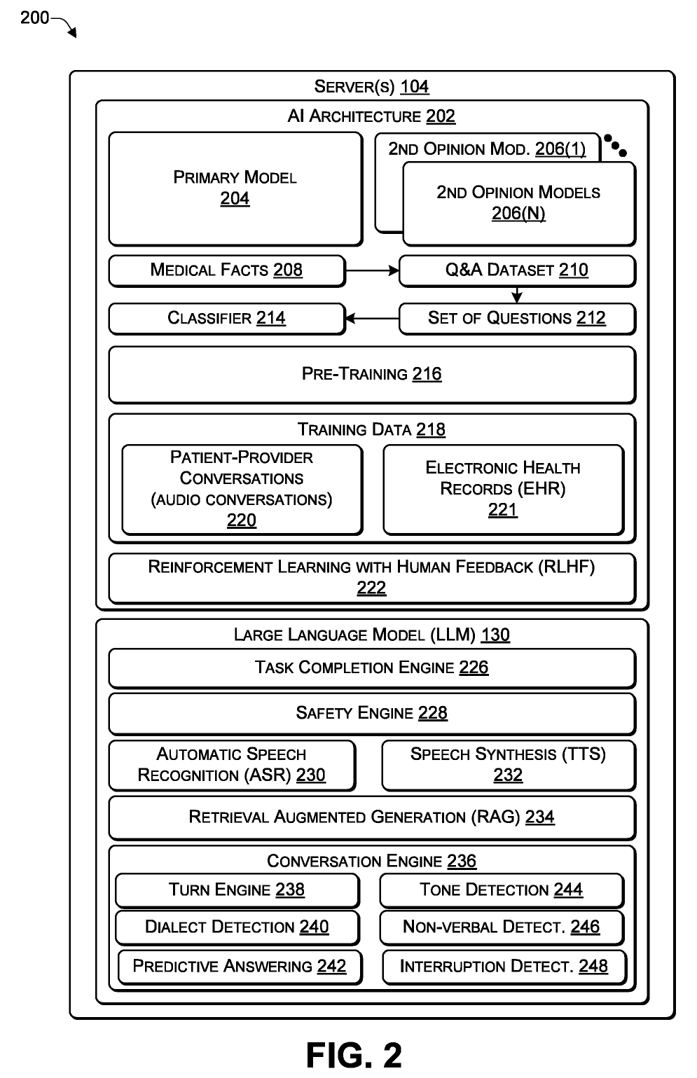

FIG. 2 is a block diagram 200 illustrating an artificial intelligence (AI) architecture 202, according to some implementations. The AI architecture 202 may be used to implement one or more of the LLMs (e.g., LLM 130(1), 130(2), 130(3), 130(4), 130(5), and 130(6) of FIG. 1) described herein.

The AI architecture 202 may include a primary model 204 which implements a primary role, such as screening, intake, scheduling, pre-op, discharge, chronic care, or any combination thereof. One or more second opinion models 206(1) to 206(N) (N>0) may be associated with the primary model 204. The second opinion models 206 are AI models, such as LLMs, that are trained to perform a more in-depth analysis than the LLM 130. Based on information extracted from the conversation 154 (of FIG. 1), one of the second opinion models 206 may perform a detailed (e.g., lengthy) analysis while the human 108 is engaged in the conversation 154 with one of the LLM 130. For example, the primary model 204 may have a 50 millisecond (ms) latency while the individual second opinion models 206 may have a 200ms latency. If the second opinion model 206 determines that particular information provided by the LLM 130 during the conversation 154 can be clarified or expanded upon, then the second opinion model 206 may provide, to the LLM 130, the particular information along with a suggestion regarding how to present the particular information to the human 108. In this way, the primary model tool for and the second opinion models 206 are part of a low-latency conversational artificial intelligence (AI) architecture with a parallelized in-depth analysis feedback loop for healthcare-related applications. The purpose of reducing latency is to prevent the human from hanging up in frustration or due to the human erroneously concluding that the conversation has concluded or the virtual assistant has encountered an error.

A large corpus of medical facts 208 are gathered and converted into a Question & Answer (Q&A) dataset 210. The Q&A dataset 210 may be used to create a set of questions 212. For example, the medical facts 208, such as a description of an illness from a textbook, and converting the description into a Q&A structure, including “What is <illness>?”, “What are the symptoms of <illness>?”, “How is <illness> treated?”, “Who usually gets <illness>?”, “Is <illness> common?”, and so on. Similarly, tables and other structured data included in the medical facts 208 are converted into a Q&A structure. One or more additional LLMs may be used to determine whether a particular question in the set of questions 212 is relevant. A classifier 214 (e.g., a support vector machine (SVM) or another type of classifier) is used to determine (predict) a distance between individual questions and their corresponding answer to determine if the answer is correct. In pre-training 216, a question is a prompt (e.g., a query presented to the models 204, 206) and the answer is the response predicted by the models 204, 206. The Q&A dataset 210 may be used to fine tune the LLM 130. For example, instruction tuning may be used to further train the LLM 130 on the Q&A dataset 210 (e.g., that is in the form of (instruction, output) pairs), in a supervised fashion.

Training data 218 includes human-provider (e.g., patient-medical professional) conversations 220 in the form of audio data and electronic healthcare records (EHR) 221. By training the LLM 130 using conversation data, the LLM 130 is more suited to engaging in the conversation 154 with the human 108. The AI architecture 202 includes providing the LLM 130 with reinforcement learning with human feedback (RLHF) in which the LLM 130 is trained using a reward function ("reward model") based on human feedback. The reward function provides the LLM 130 with a high reward for good output and a low reward for bad (poor) output. In this way, the LLM 130 is trained to provide the appropriate response at each turn in a conversation.

The LLM 130 (representing one of the LLMs 130(1) to 130(6) of FIG. 1) includes a task completion engine 226 that is tasked with completing a checklist during a particular conversation with the human. The task completion engine 226 enables the LLM 130 to complete tasks included in the checklist even while pursuing peripheral matters during the conversation, where such peripheral matters may help to build rapport between the virtual assistant and the human. For example, during the conversation with the human, the human may ask a question, such as a request for clarification of a particular topic (“Can I eat X prior to having the procedure?”). In response, the LLM 130 may provide information to answer the human’s question. The LLM may also engage in banter about favorite foods, or the human’s experiences with the foods, etc. After the LLM 130 answers the human’s question and or engages in banter, the task completion engine 226 causes the LLM 130 to resume addressing items in the checklist. The LLM 130 may include a safety engine 228 to determine a medical accuracy of the information provided by the LLM 130 to the human. The safety engine 228 may determine when to perform a “kick out” and transfer a conversation between a human and an LLM to a medical professional.

The LLM 130 includes an automatic speech recognition module (ASR) 230 to perform speech recognition of the utterances 152 provided by the human 108. The LLM 130 includes a speech synthesis module 232 to perform text-to-speech (TTS) conversion. For example, after the LLM 130 looks up text-based information, the information is provided to the human in the form of speech using the speech synthesis module 232.

The LLM 130 includes a retrieval augmented generation (RAG) module 234. The RAG 234 is an AI framework to retrieve facts from an external knowledge base to provide the LLM 130 accurate and up-to-date information. The RAG 234 grounds the LLM 130 on external sources of knowledge to supplement the LLM 130. Implementing RAG 234 in an LLM-based question answering system enables the LLM 130 to access current, reliable facts, and the access to the sources of the LLM 130, enables output of the LLM 130 to be checked for accuracy. Using RAG, the AI-based virtual assistant described herein can provide accurate answers to a large number of questions that a human could not, such as menu options for a large number of local restaurants, details for a large number of insurance policies, details about a large number of hospital facilities (such as parking information, department locations, etc.).

The LLM 130 includes the conversation engine 236. The conversation engine 236 includes a turn engine 238, dialect detection 240, predictive answering 242, tone detection 244, nonverbal detection 246, and interruption detection 248. The turn engine 238 determines when the human has completed a turn and the LLM 130 has a turn in the conversation. The dialect detection 240 detects a dialect in the utterances of the human and causes the LLM 130 to output audio data having the same (or similar) dialect. For example, the dialect detection 240 may detect the dialect based on a particular word or a particular phrase in the human’s utterances. To illustrate, the particular word or the particular phrase may be a vernacular term used in a particular geographic region.

The predictive answering 242 may predict multiple responses that the human may utter based on what the LLM 130 previously provided to the human. For example, the LLM 130 may provide a particular output (audio data) during the LLM’s turn and then listen to the human during the human’s turn. The predictive answering 242 may predict (i) possible human responses and (ii) corresponding answers based at least in part on the particular output provided during the LLM’s turn. After the human has completed providing utterances in the human’s turn, the LLM 130 may determine which of the predicted human responses the human provided and select a corresponding predicted answer. In this way, the predictive answering 242 is able to reduce the latency when providing a response after the human’s turn.

The tone detection 244 is able to detect that the tone and changes to the tone in the human’s utterances during the conversation. For example, the tone detection 244 may be able to detect a tone of the human’s utterances, correlate the tone with a particular mood, and adjust the audio data output by the LLM 130 accordingly. To illustrate, the tone of the human’s utterances may be determined based on a pitch of the utterances, a volume of the utterances, a particular word in the utterances, a particular phrase in the utterances, or any combination thereof. If a particular tone is detected, such as that of anger or frustration, then the LLM 130 may adjust the audio data output accordingly, including changing the pitch of the audio data, changing a volume of the audio data, inserting a particular word or a particular phrase in the audio data, or any combination thereof. In some cases, if the tone of the human is regressing instead of progressing (e.g., the human has become more angry or more frustrated in a subsequent turn), then the LLM 130 may offer to transfer (“kick out”) the conversation to a human. The non-verbal detection 246 may detect the human coughing, sneezing, sighing, crying, or providing another nonverbal audio cue. Detecting one of the nonverbal audio cues may cause the LLM 130 to ask additional (e.g., follow-up) questions, such as “How long have you had that cough?” (in response to detecting a cough), “How long have you had nasal congestion?” (in response to detecting a sneeze or sniffling), “Are you feeling okay?” (in response to detecting a loud sigh or crying), or the like. The interruption detection 248 may detect when the human is attempting to interrupt the LLM 130 and cause the LLM 130 to yield its turn and provide the human with a turn to enable the human to interject additional utterances into the conversation.

Thus, an AI architecture may include a primary model and one or more second opinion models to provide a low-latency conversational AI with a parallelized in-depth analysis and feedback loop in which the primary model engages in a low latency conversation with the human while the second opinion model gathers additional data and provides feedback (e.g., clarification or expansion of something that the primary model said to the human). Unlike conventional conversational AI’s, the training data includes human provider audio-based conversations in addition to text-based training data. The LLM is trained using RLHF to provide improved conversational abilities to the LLM. The LLM may use a task completion engine to avoid missing any items on a checklist of items to be covered with the human. The conversation engine used by the LLM may include a turn engine to determine when the human’s turn has ended and the LLM’s turn begins, dialect detection to understand and respond to the human in a local dialect, predictive answering to reduce latency by predicting possible answers while the human is talking, tone detection to detect emotional cues (e.g., based on a change in pitch, a change in volume, or the like) in the human’s voice, non-verbal detection (e.g., coughing, sneezing, crying, or the like), and interruption detection to detect when the human is attempting to interrupt the LLM. In this way, the LLM is able to engage in a conversation with a human in a manner similar to a human. The purpose of reducing latency is to prevent the human from hanging up in frustration or due to the human erroneously concluding that the conversation has concluded or the virtual assistant has encountered an error.

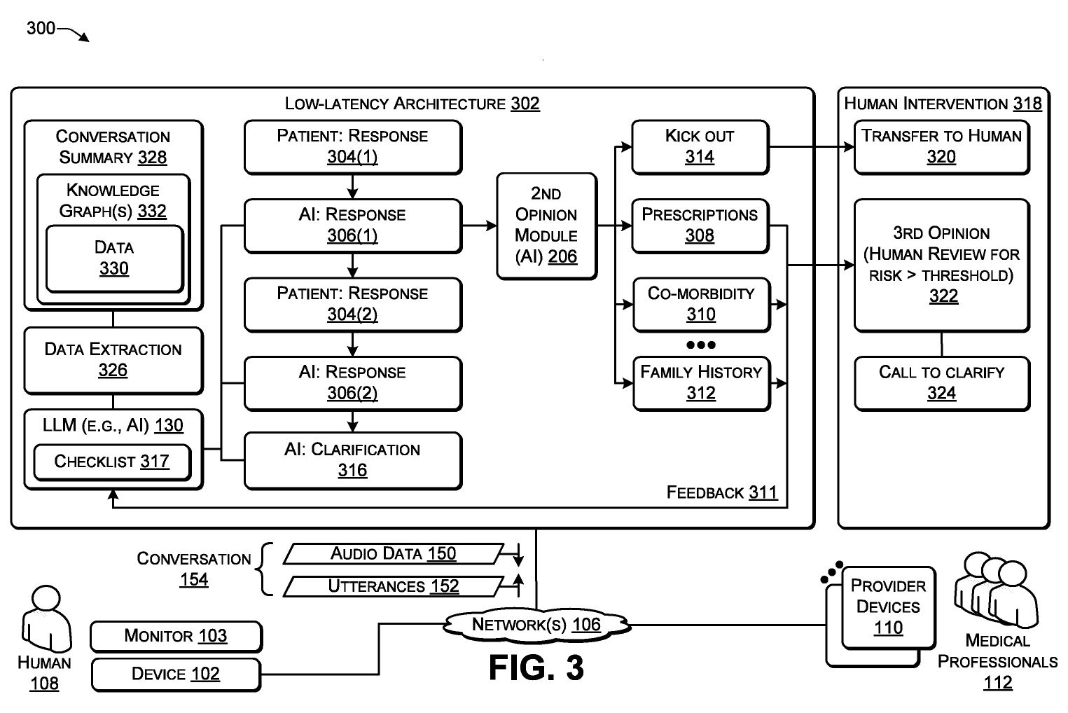

FIG. 3 is a block diagram of a system 300 that includes a low-latency conversational artificial intelligence (AI) architecture with a parallelized in-depth analysis and feedback loop, according to some implementations. The system 300 includes a low latency architecture 302 in which the LLM 130 receives a (first) response 304(1) from the human 108. The LLM 130 may engage in the conversation 154 with the human 108 based on a checklist 317 that identifies tasks that the LLM 130 is to complete during the conversation 154. The LLM 130 provides a (first) response 306(1) to the human 108. The LLM 130 receives a (second) response 304(2) from the human 108 and provides a (second) response 306(2) to the human 108. While the LLM 130 is engaged in the conversation 154 with the human 108, the second opinion module 206 may, in parallel (substantially at the same time), do a lengthier “deep dive” by performing research into the human response 304(1) and the AI response 306(1). For example, the research may include prescriptions 308, co-morbidity 310, family history 312 and the like. The prescriptions 308 may include determining whether the human 108 is being prescribed prescriptions appropriate to the human’s conditions, determining whether the human 108 is being prescribed the correct dosages of the prescriptions, determining whether the prescribed prescriptions have any undesirable interactions or contraindications, and other prescription related information. The prescriptions 308 may include a dosage engine that is invoked every time there is mention of a drug, dosage, or both. The co-morbidity 310 may include determining whether the human 108 has two or more medical conditions that may be related to an underlying cause or that have an undesirable interaction. The family history 312 may include determining whether the human’s parents, siblings, or other relatives have a history of medical conditions similar to that of the human 108.

Based on determining the research into the prescriptions 308, the co-morbidity 310, and the family history 312, the second opinion module 206 may determine whether the AI response 306(1) was appropriate or whether the AI response 306(1) may be clarified. If the second opinion module 206 determines that the response 306(1) is appropriate, the second opinion module 206 does not take further action. If the second opinion module 206 determines that the response 306(1) may be clarified, then the second opinion module 206 may provide feedback 311 to the LLM 130, causing the LLM 130 to provide a clarification 316. For example, the human response 304(1) may be “Can I have soup?” and the AI response 306(1) may be “yes, you can have soup.”. The clarification 316 may be “I previously said you could have soup. I noticed that either you or at least one of your family members have high blood pressure, so please check the sodium content of any pre-packaged soups and avoid soups with high sodium.” In this example, “you … have high blood pressure” was determined by checking co-morbidity 310 and “at least one of your family members have high blood pressure” was determined by checking family history 312. As another example, the clarification 316 may be “I noticed that you are currently taking <1st prescription> in the morning. Instead of taking <2nd prescription> in the morning as I previously suggested, I suggest taking <2nd prescription> in the evening, around 12 hours after taking <1st prescription>.” In this example, by checking the prescription 308, the second opinion module 206 may determine that the patient is taking the 1st prescription and that the 2nd prescription (mentioned in the AI response 306(1)) is not to be taken at the same time as the 1st prescription and instruct the LLM 130 to clarify when to take the 2nd prescription. As a further example, the clarification 316 may be “I noticed that your potassium was high in your most recent blood work. While I had previously suggested eating more avocados (for the oil), I suggest not having more than one avocado a week to avoid raising your potassium further, as avocados are high in potassium”. In this example, by checking the co-morbidity 310 that includes test results (such as the results of a blood workup, e.g., comprehensive metabolic panel), the second opinion module 206 may determine that the patient has a high potassium level and that avocados are high in potassium and clarify how many avocados the human can have in a particular time frame.

In some cases, the second opinion module 206 may determine that providing the clarification 316 is insufficient and that the conversation 154 would benefit from human intervention 318. In such cases, the second opinion module 206 may use a kick out 314 engine to determine to transfer the conversation 154 to the medical professional 320 (e.g., human nurse or doctor). In addition, if the second opinion module 206 determines that a risk to the human 108 is greater than a predetermined threshold, then the second opinion module 206 may initiate a request for a third opinion 322 in which a human (e.g., a doctor, such as a specialist) reviews the human’s case and takes appropriate action (e.g., provides a call (to the human 108) to clarify 324 the situation). The kick out engine will be invoked for qualitative statements that suggest symptoms that require further evaluation.

While the LLM 130 is engaged in the conversation 154 (e.g., the responses 304, 306), one or more background processes may perform data extraction 326 to extract data 330 (e.g., pertinent information, such as facts) to create a conversation summary 328 which is stored in the form of a knowledge graph 332 (or another type of data structure). The conversation summary 328 and the data 330 may enable the LLM 130 to quickly access earlier information in a lengthy conversation. With medical-related AI, conversations may be lengthy and involve many turns (e.g., 50 to 100 turns is common and the conversation may, in some cases, include 200 or even 300 turns). In such lengthy conversations, the human 108 may reference previously provided information (“referenced information”) in a current response. If the referenced information was provided early in the conversation, a conventional virtual assistant may have “forgotten” the referenced information and thus provide responses that are frustrating to the human. Alternatively, a conventional virtual assistant may have to scan through a transcript of the conversation to determine the referenced information. Such a scan may cause a lengthy delay and introduce latency into the conversation 154. By performing data extraction 326 and creating the conversation summary 328 and maintaining the knowledge graph 332 storing the data 330, the LLM 130 is able to quickly access the information (data 330) referenced by the human, thereby reducing latency. The purpose of reducing latency is to provide a natural sounding conversation and prevent the human from hanging up (i) in frustration or (ii) due to the human erroneously concluding that the conversation has concluded or the virtual assistant has encountered an error.

Thus, a low latency architecture for a conversational AI includes an AI, such as an LLM, engaged in a conversation with the human. While the AI is engaged in the conversation with the human, a second opinion module does a more in-depth analysis of information provided by the human. If the second opinion module determines that a response provided by the conversational AI is to be clarified, the second opinion module provides feedback that the conversational AI uses to provide a clarification to the human during the conversation. By parallelizing the second opinion module with the conversational AI, latency is reduced, thereby making the conversation more natural. In addition, while the conversational AI is engaged in the conversation with the human, a data extraction module (in parallel) extracts facts from each human response and creates a conversation summary in the form of a data structure that can be quickly searched, such as a knowledge graph. The conversational AI can quickly access the conversation summary to look up information previously provided by the human that the human is referencing in a current turn in the conversation. The access to the conversation summary enables the conversational AI to reduce latency because the conversation AI is not pausing the conversation to perform a search of a transcript of the conversation.

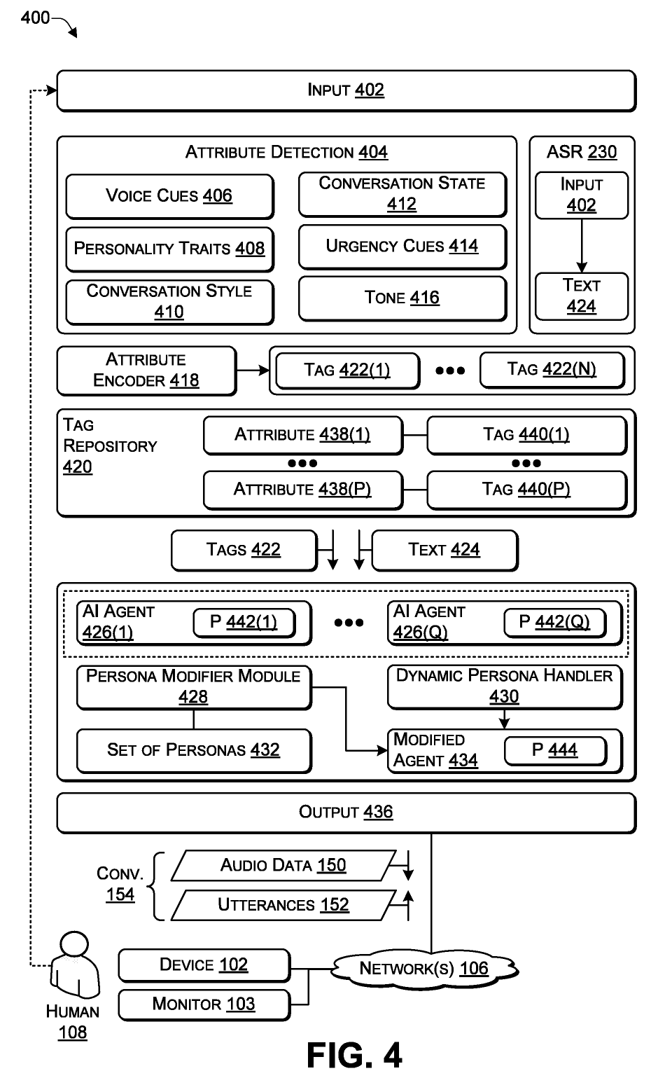

FIG. 4 is a block diagram of a system 400 to determine attributes of human input and modify a persona of an AI-based agent based on the attributes, according to some implementations. The system 400 receives input 402 from the human 108. For example, the input 402 may be audio that includes speech. In some cases, at least a portion of the attribute detection 404 may occur before the ASR 230 converts the input 402 into speech 424. In other cases, at least a portion of the attribute detection 404 may occur after the ASR 230 converts the input 402 into speech 424. In still other cases, at least a portion of the attribute detection 404 may occur in parallel with the ASR 230 converting the input 402 into speech 424. For example, based on the ASR 230 and the attribute detection 404, diarization may be performed by using timestamps on utterances to back-calculate cadence, hesitation, and the like. In addition, word choice, including an emotional state based on word choice may also be determined while ASR 230 is being performed or after the ASR 230 has been completed.

Attribute detection 404 may analyze the input 402 and/or the text 424 to determine various speech attributes, including, for example, voice cues 406, personality traits 408, conversation style 410, conversation state 412, urgency cues 414 and tone 416. The voice cues 406 may include, for example, hesitation, rising pitch, breathlessness, energy level, speech rate, interruptions (the human 108 interrupts the AI agent while the AI agent is speaking), and the like. The personality traits 408 may include, for example, verbose, concise, empathetic, familiar, tangent-oriented, and the like. The conversation style 410 may include, for example, slow-paced, fast-paced, structured, and the like. The conversation state 412 may include, for example, attentive, frustrated, confused, disengaged, distracted, and the like. The urgency cues 414 may include, for example, rapid speech, repeated queries, explicit time constraints (e.g., the human 108 includes in the input 402 “I only have a few minutes”, “I need to leave soon”, or the like). The tone 416 may include the vocal quality (e.g., smooth or hoarse), pitch (e.g., normal, higher than normal), volume (e.g., normal, lower than normal, higher than normal), and pace (speed at which words are spoken, gaps between words) that convey emotion, attitude, and intent.

An attribute encoder 418 may encode the various attributes 406, 408, 410, 412, 414, and 416 that the attribute encoder 418 has identified in the input 402 and/or text 424 to create one or more tags 422(1) to 422(N) (N>0). The tags 422 thus encode the various attributes that attribute detection 404 identified in the input 402. The attribute encoder 418 may access a tag repository 420 to identify a tag corresponding to a particular attribute. For example, the tag repository 420 may include attributes 438(1) to 438(P) (P>0) and corresponding tags 440(1) to 440(P). After identifying a particular attribute, the attribute encoder 418 may identify the corresponding tag (e.g., tag 440(P) corresponding to attribute 438(P)) in the tag repository 420.

The automatic speech recognition (ASR) 230 may convert the input 402 into text 424. The text 424 is provided to one of the AI agents 426(1) to 426(Q) that will engage in a conversation with the human 108. Based on the tags 422, the persona modifier module 428 and the dynamic persona handler 430 may modify a persona of one of the AI agents 426 that is engaged in a conversation with the human 108 to create a modified agent 434. The persona modifier module 428 may access a set of personas 432 and select one or more personas from the set of personas 432 based on the tags 422. Each AI agent 426 may have an initial persona. For example, AI agent 426(1) may have a persona 442(1) and AI agent 426(Q) may have a persona 442(Q).

The persona modifier module 428 may map a set of incoming (e.g., most recently determined) tags 422 to latent behavioral representations (the set of personas 432) and apply them in real time to modify the persona 442 of one of the AI agents 426 to create the modified agent 434 with a modified persona 444. The persona modifier module 428 may perform tag stacking (modifying a persona of an AI agent based on two or more of the tags 422), tag blending (modifying a persona of an AI agent based on blending two or more of the tags 422), tag priority (weighting the tags 422 based on priority, with higher priority tags getting a larger weight than lower priority tags), decaying (aging out) older tags (e.g., tags may be aged out after a predetermined number of turns or after a predetermined period of time), and real time persona modification without disrupting coherence when switching from one persona to another persona.

The dynamic persona handler 430 may determine when and how a persona shifts (from one persona to another persona) based on: (1) voice cues 406, such as a tone of voice, a tempo of speech, interjections, and stress markers, such as sighs, (2) urgency cues 414, such as rapid speech, repeated queries, and explicitly expressed time constraints, (3) engagement cues, such as silence patterns and disengagement markers, (4) user archetype detection, such as cautious, assertive, novice, expert, anxious, and the like, and (5) conversation goals, such as escalation, de-escalation, clarification, and task completion.

The modified agent 434 with the persona 444 may provide output 436 that is output as audio data 150 during the conversation 154. The process of (i) receiving input 402 from the human 108, (ii) determining attributes 406, 408, 410, 412, 414, 416, (iii) determining tags 422 corresponding to the attributes, and (iv) modifying a persona 442 of one of the agents 426 to create the modified agent 434 that responds to the input 402 by providing the output 436 may be repeated until the modified agent 434 determines that the conversation can be ended (e.g., typically after the modified agent 434 determines that the checklist 317 has been completed). In this way, the persona of an AI agent that is engaged in the conversation 154 with the human 108 is dynamically and adaptively modified based on attributes of the input 402 from the human 108.

Assume an initial persona of an AI agent speaks with an initial cadence. The cadence of the words spoken includes varying (i) a pitch, (ii) a volume, (iii) a speed, and (iv) pause lengths. The modified persona may use a modified cadence where one or more of the (i) pitch, (ii) volume, (iii) speed, and (iv) pause lengths are different from the initial cadence. For example, if the system detects urgency in the input 402, then the volume may fluctuate less, the speed may increase, the pause lengths may decrease, or any combination thereof. If the system detects anxiousness in the input 402, then the cadence may be modified to a more calming cadence in which the volume may fluctuate less, the speed may decrease, the pause lengths may increase, or any combination thereof. If the system detects exuberance in the input 402, then the cadence may be modified to a cadence in which the volume may fluctuate more, the speed may increase, the pause lengths may increase, or any combination thereof. For example, a word in a phrase or a sentence may be emphasized by (i) increasing a pause before the word, (ii) increasing a volume of the word, (iii) increasing or decreasing a pitch at which the word is spoken, or (iv) any combination thereof.

In some cases, each persona may have an associated set of cadences. Each cadence may have a predefined sequence in which to pronounce a sentence or a phrase. Each predefined sequence may have a particular speed at which the sentence or phrase is to be spoken, a first pitch and a first volume for a first word (of the sentence or phrase), a length of a first pause between the first word and the second word, a second pitch and a second volume for a second word (of the sentence or phrase), a length of a second pause between the second word and the third word, and so on. Each cadence may be designed for a particular purpose. For example, a cadence with a faster speed and higher pitch and less volume variation for each word may be used when urgency cues are detected. As another example, a cadence with a slower speed and lower pitch and more volume variation for each word (with a higher volume emphasizing key words) may be used when cues are detected indicating that the human is confused or frustrated. In such cases, the cadence may be designed to calm the human.

A cadence is a set of characteristics associated with speaking multiple words (e.g., a phrase or a sentence). The cadence specifies a speed at which multiple words are output, a pitch at which individual words of the multiple words are output, a volume at which individual words are output, a length of each pause between two consecutive words (with the pause varying between a first word and a second word and between the second word and a third word, and so on), and the like. The differences between the pitch, volume, and pause length may vary slightly between words. For example, the pitch may vary a few microtones to a one or more semitones, the volume may vary fractions of a decibel, the pause length may vary by a few tenths to a few one hundredths of a second, and so on.

For example, for five words, two different cadences may be as follows: persona 1 uses cadence 1: speed 1 (word 1, pitch 1, volume 1), pause 1, (word 2, pitch 2, volume 2), pause 2, (word 3, pitch 3, volume 3), pause 3, (word 4, pitch 4, volume 4), pause 4, (word 5, pitch 5, volume 5) persona 2 uses cadence 2: speed 2 (word 1, pitch 6, volume 6), pause 1, (word 2, pitch 7, volume 7), pause 2, (word 3, pitch 8, volume 8), pause 3, (word 4, pitch 9, volume 9), pause 4, (word 5, pitch 10, volume 10)

In this example, cadence 1 may be modeled after the cadence of a woman and cadence 2 may be modeled after the cadence of a man. In some cases, the cadence may include an accent, such as Southern United States, Australian, Scottish, Irish, British, Latin, North Indian, South Indian, Bengali, Gujrati, Chinese, or another type of accent.

Thus, an AI agent may engage in a conversation with a human and receive an initial input. A system in which the AI agent operates may determine attributes associated with the input, determine tags corresponding to the attributes, and modify a persona of the AI agent, based on the tags, to create a modified agent with a modified persona. The modified agent may respond to the initial input by providing output. The system receives a next input from the human, determines a next set of attributes associated with the next input, determines a next set of tags associated with the next set of attributes, and modifies the modified agent’s persona based on the next set of tags. This process may be repeated until the modified agent determines that the conversation can be ended (e.g., task checklist has been completed or human has provided cues indicating a desire to end the conversation). In this way, the persona of an AI agent that is engaged in a conversation with a human is dynamically and adaptively modified based on attributes of each input provided by the human, resulting in up to a 20% improvement in call completions.

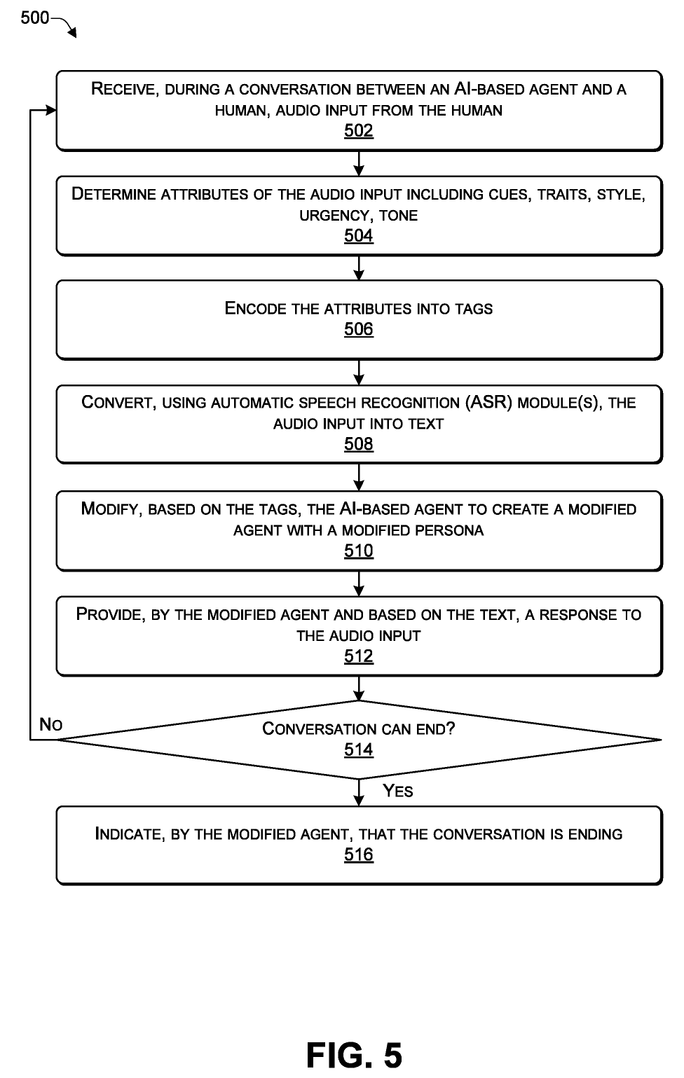

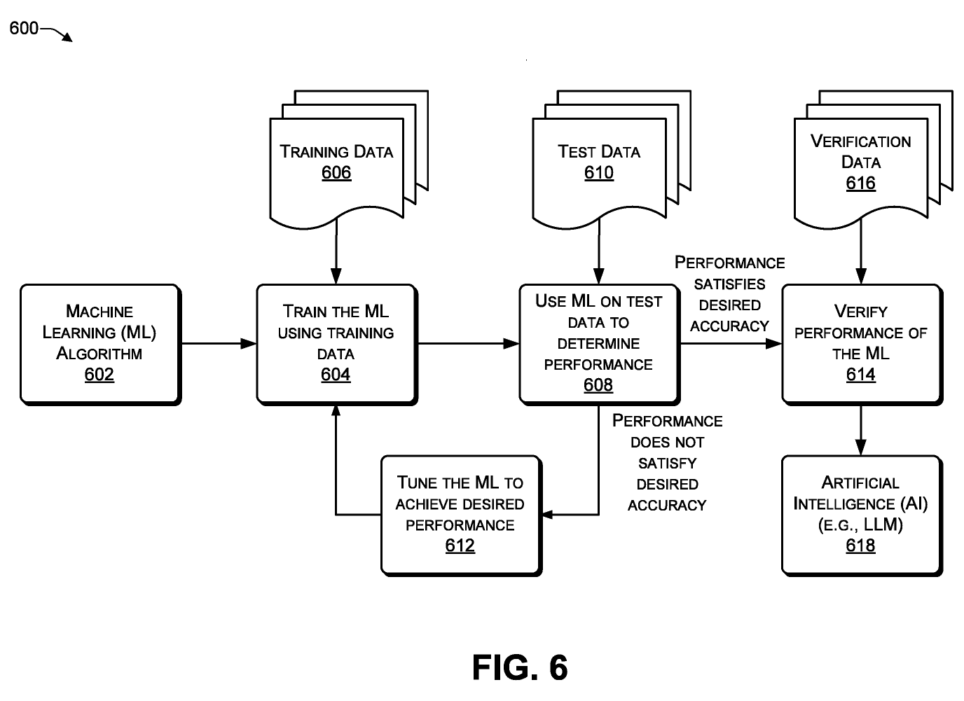

In the flow diagram of FIGS. 5 and 6, each block represents one or more operations that can be implemented in hardware, software, or a combination thereof. In the context of software, the blocks represent computer-executable instructions that, when executed by one or more processors, cause the processors to perform the recited operations. Generally, computer-executable instructions include routines, programs, objects, modules, components, data structures, and the like that perform particular functions or implement particular abstract data types. The order in which the blocks are described is not intended to be construed as a limitation, and any number of the described operations can be combined in any order and/or in parallel to implement the processes. For discussion purposes, the processes 500 and 600 are described with reference to FIGS. 1, 2, 3, and FIG. 4 as described above, although other models, frameworks, systems and environments may be used to implement these processes.

FIG. 5 is a flowchart of a process 500 that includes determining attributes of an audio input received from a human, according to some implementations. The process 500 may be performed by one or more components of the system 400 of FIG. 4.

At 502, the process may, during a conversation between an AI-based agent and a human, receive audio input from the human. At 504, the process may determine attributes of the audio input, including cues, traits, style, urgency, tone, and the like. At 506, the process may encode the attributes into a set of (one or more) tags. For example, in FIG. 4, attribute detection 404 may receive the input 402 from the human 108 and determine one or more attributes 406, 408, 410, 412, 414, 416. The attribute encoder 418 may determine, using the tag repository 420, the tags 422(1) to 422(N) to encode the attributes 406, 408, 410, 412, 414, 416.

At 508, the process may convert, using one or more automatic speech recognition (ASR) modules, the audio input into text. At 510, the process may modify the AI-based agent to create a modified agent with the modified persona based at least in part on the tags. At 512, the modified agent may provide a response to the audio input based on the text. For example, in FIG. 4, one or more automatic speech recognition (ASR) modules 230 may convert the input 402 into the text 424. The persona modifier module 428 may modify one of the AI agents 426 having a persona 442 based on the tags 422 to create the modified agent 434 having the modified persona 444.

At 514, the process may determine whether the conversation can be ended or is ending. If a determination is made, at 514, that the conversation is not ending (the conversation is to continue), then the process may proceed back to 502 to receive additional audio input from the human. In this way, 502, 504, 506, 508, 510, 512, and 514 may be repeated until the process determines that the conversation can be ended or is ending. If a determination is made, at 514 that the conversation can be ended or is ending, the process may indicate, by the modified agent, that the conversation is ending, at 516. For example, in FIG. 4, if the modified agent 434 determines, based on the text 424, that the conversation 154 can be ended (e.g., because the checklist 317 has been completed) or because the human 108 has provided cues in the input 402 that the human 108 desires to end the conversation 154, then the modified agent 434 may end the conversation 154. If the modified agent 434 determines, based on the text 424, that the conversation 154 is to continue, then the system may wait for the human 108 to provide a next input.

Thus, a conversation between an AI agent and a human may be initiated. As part of the conversation, the human may provide an initial input. Attributes associated with the input may be determined and tags corresponding to the attributes may be determined. The tags may be used to modify a persona of the AI agent, to create a modified agent with a modified persona. The modified agent may respond to the initial input by providing output. In response, the human may provide a next input. The process of determining a next set of attributes associated with the next input, determining a next set of tags associated with the next set of attributes, and modifying the agent’s persona based on the next set of tags may be repeated until the agent determines that the conversation has been completed and can be ended. In this way, the persona of an AI agent that is engaged in a conversation with a human is dynamically and adaptively modified based on attributes of each input provided by the human, resulting in up to a 20% improvement in call completions.

FIG. 6 is a flowchart of a process 600 to train a machine learning algorithm (to create an AI), according to some implementations. For example, the process 600 may be performed during the pre-training 216 or other training described herein to create any one of the AI (including ASR) and LLMs described herein.

At 602, a machine learning algorithm (e.g., software code) may be created by one or more software designers. For example, the LLMs 130 may be created by software designers. At 604, the machine learning algorithm may be trained using pre-classified training data 606. For example, the training data 606 may have been pre-classified by humans, by machine learning, or a combination of both. After the machine learning has been trained using the pre-classified training data 606, the machine learning may be tested, at 608, using test data 610 to determine a performance metric of the machine learning. The performance metric may include, for example, precision, recall, Frechet Inception Distance (FID), or a more complex performance metric. For example, in the case of a classifier, the accuracy of the classification may be determined using the test data 610.

If the performance metric of the machine learning does not satisfy a desired measurement (e.g., 95%, 98%, 99% in the case of accuracy), at 608, then the machine learning code may be tuned, at 612, to achieve the desired performance measurement. For example, at 612, the software designers may modify the machine learning software code to improve the performance of the machine learning algorithm. After the machine learning has been tuned, at 612, the machine learning may be retrained, at 604, using the pre-classified training data 606. In this way, 604, 608, 612 may be repeated until the performance of the machine learning is able to satisfy the desired performance metric. For example, in the case of a classifier, the classifier may be tuned to be able to classify the test data 610 with the desired accuracy.

After determining, at 608, that the performance of the machine learning satisfies the desired performance metric, the process may proceed to 614, where verification data 616 may be used to verify the performance of the machine learning. After the performance of the machine learning is verified, at 614, the machine learning 602, which has been trained to provide a particular level of performance may be used as an artificial intelligence (AI) 618, such as any of the AI or LLM described herein.

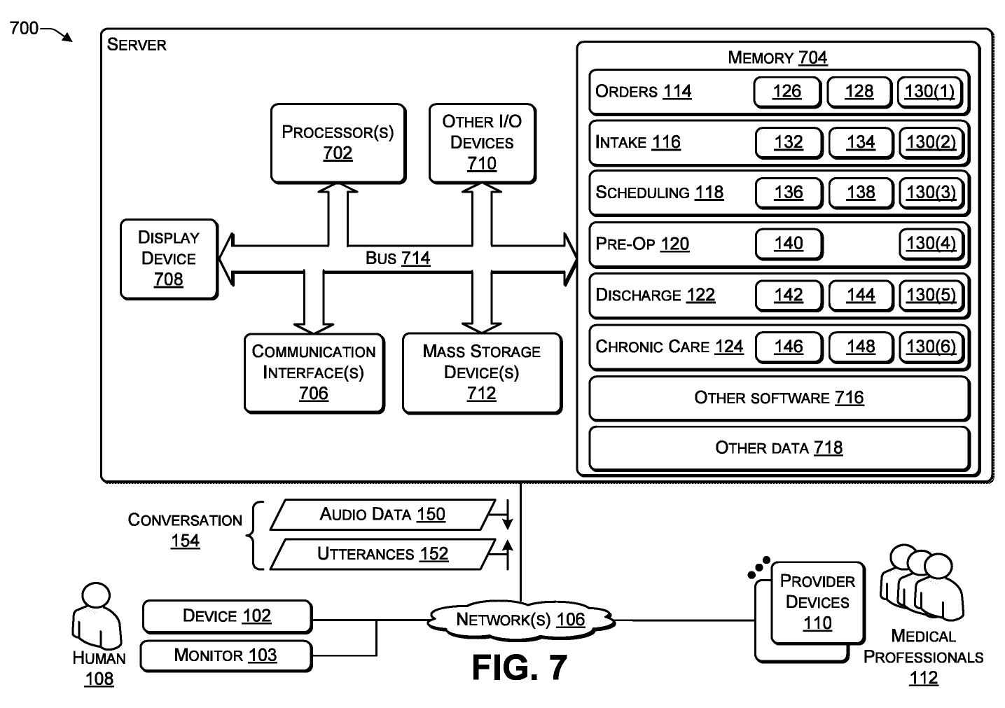

FIG. 7 illustrates an example configuration of a device 700 that can be used to implement the systems and techniques described herein. For example, the device 700 may be a server (or a set of servers) used to host one or more of the components described herein. In some cases, the systems and techniques described herein may be implemented as an application programming interface (API), a plugin, or another type of implementation.

The device 700 may include one or more processors 702 (e.g., central processing unit (CPU), graphics processing unit (GPU), or the like), a memory 704, communication interfaces 706, a display device 708, other input/output (I/O) devices 710 (e.g., keyboard, trackball, and the like), and one or more mass storage devices 712 (e.g., disk drive, solid state disk drive, or the like), configured to communicate with each other, such as via one or more system buses 714 or other suitable connections. While a single system bus 714 is illustrated for ease of understanding, it should be understood that the system bus 714 may include multiple buses, such as a memory device bus, a storage device bus (e.g., serial ATA (SATA) and the like), data buses (e.g., universal serial bus (USB) and the like), video signal buses (e.g., ThunderBolt®, digital video interface (DVI), high definition media interface (HDMI), and the like), power buses, etc.

The processors 702 are one or more hardware devices that may include a single processing unit or a number of processing units, all of which may include single or multiple computing units or multiple cores. The processors 702 may include a graphics processing unit (GPU) that is integrated into the CPU or the GPU may be a separate processor device from the CPU. The processors 702 may be implemented as one or more microprocessors, microcomputers, microcontrollers, digital signal processors, central processing units, graphics processing units, state machines, logic circuitries, and/or any devices that manipulate signals based on operational instructions. Among other capabilities, the processors 702 may be configured to fetch and execute computer-readable instructions stored in the memory 704, mass storage devices 712, or other computer-readable media.

Memory 704 and mass storage devices 712 are examples of computer storage media (e.g., memory storage devices) for storing instructions that can be executed by the processors 702 to perform the various functions described herein. For example, memory 704 may include both volatile memory and non-volatile memory (e.g., random access memory (RAM), read only memory (ROM), or the like) devices. Further, mass storage devices 712 may include hard disk drives, solid-state drives, removable media, including external and removable drives, memory cards, flash memory, floppy disks, optical disks (e.g., compact disc (CD), digital versatile disc (DVD), a storage array, a network attached storage (NAS), a storage area network (SAN), or the like. Both memory 704 and mass storage devices 712 may be collectively referred to as memory or computer storage media herein and may be any type of non-transitory media capable of storing computer-readable, processor-executable program instructions as computer program code that can be executed by the processors 702 as a particular machine configured for carrying out the operations and functions described in the implementations herein.

The device 700 may include one or more communication interfaces 706 for exchanging data via the network 106. The communication interfaces 706 can facilitate communications within a wide variety of networks and protocol types, including wired networks (e.g., Ethernet, Data Over Cable Service Interface Specification (DOCSIS), digital subscriber line (DSL), Fiber, universal serial bus (USB) etc.) and wireless networks (e.g., wireless local area network (WLAN), global system for mobile (GSM), code division multiple access (CDMA), 802.11, Bluetooth, Wireless USB, ZigBee, cellular, satellite, etc.), the Internet and the like. Communication interfaces 706 can also provide communication with external storage, such as a storage array, network attached storage, storage area network, cloud storage, or the like.

The display device 708 may be used for displaying content (e.g., information and images) to users. Other I/O devices 710 may be devices that receive various inputs from a user and provide various outputs to the user, and may include a keyboard, a touchpad, a mouse, a gaming controller (e.g., joystick, steering controller, accelerator pedal, brake pedal controller, virtual reality (VR) headset, VR glove, or the like), a printer, audio input/output devices, and so forth.

The computer storage media, such as memory 704 and mass storage devices 712, may be used to store any of the software and data described herein. The example systems and computing devices described herein are merely examples suitable for some implementations and are not intended to suggest any limitation as to the scope of use or functionality of the environments, architectures and frameworks that can implement the processes, components and features described herein. Thus, implementations herein are operational with numerous environments or architectures, and may be implemented in general purpose and special-purpose computing systems, or other devices having processing capability. Generally, any of the functions described with reference to the figures can be implemented using software, hardware (e.g., fixed logic circuitry) or a combination of these implementations. The term “module,” “mechanism” or “component” as used herein generally represents software, hardware, or a combination of software and hardware that can be configured to implement prescribed functions. For instance, in the case of a software implementation, the term “module,” “mechanism” or “component” can represent program code (and/or declarative-type instructions) that performs specified tasks or operations when executed on a processing device or devices (e.g., CPUs or processors). The program code can be stored in one or more non-transitory computer-readable memory devices or other computer storage devices. Thus, the processes, components and modules described herein may be implemented by a non-transitory computer program product.

1. A method, comprising:
engaging, by an artificial intelligence agent executed by one or more processors, in a conversation with a human;
receiving, by the artificial intelligence agent, a first audio input from the human;
determining, by the one or more processors, a first set of attributes of the first audio input;
encoding the first set of attributes, by the one or more processors, to create a first set of tags, individual attributes of the first set of attributes having a corresponding tag in the first set of tags;
modifying, by the one or more processors and based on the first set of tags, a persona of the artificial intelligence agent to create a first modified agent having a first modified persona; and
providing, by the first modified agent having the first modified persona, a first response to the human.

2. The method of claim 1, further comprising:
detecting an urgency signal in the first audio input based at least in part on:
a linguistic marker comprising at least one particular word in the first audio input;
an acoustic marker comprising at least one of:
a speed of speech included in the first audio input;
a length of one or more pauses in the first audio input;
a pitch of one or more words in the first audio input; or
any combination thereof; or
both the linguistic marker and the acoustic marker.

3. The method of claim 1, wherein the first set of attributes of the first audio input comprise:
a voice cue;
a personality trait;
an urgency cue;
a conversation style;
a conversation state;
a tone; or
any combination thereof.

4. The method of claim 1, further comprising:
providing the first audio input to an automatic speech recognition module;
receiving a text output from the automatic speech recognition module that is derived from the first audio input.

5. The method of claim 1, further comprising:
receiving, by the first modified agent having the first modified persona, a second audio input from the human;
determining a second set of attributes of the second audio input;
encoding the second set of attributes into a second set of tags;
modifying, based on the second set of tags, the first modified persona of the first modified agent to create a second modified agent having a second modified persona; and
providing, by the second modified agent having the second modified persona, a second response to the human.

6. The method of claim 5, further comprising:
during the conversation with the human, maintaining coherency when transitioning from the first modified agent having the first modified persona to the second modified agent having the second modified persona.

7. The method of claim 5, wherein:
the first response provided by the first modified agent having the first modified persona uses a first cadence; and
the second response provided by the second modified agent having the second modified persona uses a second cadence in which at least one word has:
a different pitch than the first cadence;
a different volume than the first cadence;
a different length of a pause after the at least one word than the first cadence; or
any combination thereof.

8. A server comprising:
one or more processors; and
one or more computer-readable storage media to store instructions executable by the one or more processors to perform operations comprising:
engaging, by an artificial intelligence agent executed by the one or more processors, in a conversation with a human;
receiving, by the artificial intelligence agent, a first audio input from the human;
determining a first set of attributes of the first audio input;
encoding the first set of attributes to create a first set of tags, individual attributes of the first set of attributes having a corresponding tag in the first set of tags;
modifying, based on the first set of tags, a persona of the artificial intelligence agent to create a first modified agent having a first modified persona; and
providing, by the first modified agent having the first modified persona, a first response to the human.

9. The server of claim 8, wherein the first modified persona comprises:
a verbose persona that provides additional details about one or more topics in the conversation;
a concise persona that does not provide the additional details about the one or more topics in the conversation;
an empathetic persona that mirrors a speaking style of the human;
a calming persona that is designed to invoke a calming response in the human; or
any combination thereof.

10. The server of claim 8, wherein the first set of attributes comprise:
a tone including formal, casual, warm, authoritative, or any combination thereof;
a personality trait including witty, concise, empathetic, familiar, tangent-oriented, or any combination thereof;
a conversation style including slow-paced, fast-paced, highly structured, or any combination thereof;
a behavioral cue including reassuring, directive, reflective, or any combination thereof;
an urgency level including urgent or non-urgent; or
any combination thereof.

11. The server of claim 8, the operations further comprising:
determining a conversation state of the conversation, the conversation state comprising one of:
engagement;
disengagement;
frustration; or
confusion.

12. The server of claim 8, the operations further comprising:
receiving, by the first modified agent having the first modified persona, a second audio input from the human;
determining a second set of attributes of the second audio input;
encoding the second set of attributes into a second set of tags;
modifying, based on the second set of tags, the first modified persona of the first modified agent to create a second modified agent having a second modified persona; and
providing, by the second modified agent having the second modified persona, a second response to the human.

13. The server of claim 12, the operations further comprising:
during the conversation with the human, maintaining coherency when transitioning from the first modified agent having the first modified persona to the second modified agent having the second modified persona.

14. The server of claim 12, wherein:
the first response provided by the first modified agent having the first modified persona uses a first cadence; and
the second response provided by the second modified agent having the second modified persona uses a second cadence in which at least one word has:
a different pitch than the first cadence;
a different volume than the first cadence;
a different length of a pause after the at least one word than the first cadence; or
any combination thereof.

15. A non-transitory memory device to store instructions executable by one or more processors to perform operations comprising:
engaging, by an artificial intelligence agent executed by the one or more processors, in a conversation with a human;
receiving, by the artificial intelligence agent, a first audio input from the human;
determining a first set of attributes of the first audio input;
encoding the first set of attributes to create a first set of tags, individual attributes of the first set of attributes having a corresponding tag in the first set of tags;
modifying, based on the first set of tags, a persona of the artificial intelligence agent to create a first modified agent having a first modified persona; and
providing, by the first modified agent having the first modified persona, a first response to the human.

16. The non-transitory memory device of claim 15, wherein the first modified persona comprises at least one of:
an empathy level that is different from the persona of the artificial intelligence agent;
a conciseness level that is different from the persona of the artificial intelligence agent;
a familiarity level that is different from the persona of the artificial intelligence agent;
a structured speech level that is different from the persona of the artificial intelligence agent;
an urgency-handling mode that is different from the persona of the artificial intelligence agent;
a knowledge density level that is different from the persona of the artificial intelligence agent; or
any combination thereof.

17. The non-transitory memory device of claim 15, the operations further comprising:
determining one or more voice cues in the first audio input including one or more of:
an amount of hesitation between words spoken in the first audio input;
a rising pitch between a word and a subsequent word in the first audio input;
an amount of breath noise detected in the first audio input;
a speech rate of the words spoken in the first audio input; or
any combination thereof.

18. The non-transitory memory device of claim 15, the operations further comprising:
receiving, by the first modified agent having the first modified persona, a second audio input from the human;
determining a second set of attributes of the second audio input;
encoding the second set of attributes into a second set of tags;
modifying, based on the second set of tags, the first modified persona of the first modified agent to create a second modified agent having a second modified persona; and
providing, by the second modified agent having the second modified persona, a second response to the human.

19. The non-transitory memory device of claim 18, wherein:
the second response provided by the second modified agent having the second modified persona includes at least one word having:
a different pitch than words included in the first response;
a different volume than the words included in the first response;
a different length of a pause after the at least one word than pauses included in the first response; or
any combination thereof.

20. The non-transitory memory device of claim 15, the operations further comprising:
detecting an urgency signal in the first audio input based at least in part on:
a linguistic marker comprising a particular word in the first audio input;
an acoustic marker comprising at least one of:
a speed of speech included in the first audio input;
a length of one or more pauses in the first audio input;
a pitch of one or more words in the first audio input; or
any combination thereof; or
both the linguistic marker and the acoustic marker.
